(19) United States
(12) Plant Patent Application Publication
(10) Pub. No.: US 2006/0195956 P1
(43) Pub. Date: Aug. 31, 2006

(54) Chrysanthemum plant named 'DANAT213'

(75) Inventor: Gabriel Danziger, Moshav Nir-Zvi (IL)

Correspondence Address: FOLEY AND LARDNER LLP;SUITE 500, 3000 K STREET NW, WASHINGTON, DC, 20007, US

(73) Assignee: Danziger "Dan" Flower Farm

(21) Appl. No.: 11/202,040

(22) Filed: Aug. 12, 2005

(30) Foreign Application Priority Data
Feb. 25, 2005 (CO) A05891

(51) Int. Cl. A01H 5/00

(52) U.S. Cl. PLT/289

(57) ABSTRACT

A new and distinct Chrysanthemum plant named ‘Danat213’ characterized by having inflorescence with oblong shaped, yellow ray florets, and bell shaped, yellow disc florets; flowering time of 7.5 weeks; short day plant; and vigorous growth habit.

### BOTANICAL DESIGNATION

Chrysanthemum morifolium

### VARIETY DENOMINATION

‘Danat213’

### BACKGROUND OF THE INVENTION

The present invention relates to a new and distinct cultivar of Chrysanthemum plant, botanically known as Chrysanthemum morifolium, hereinafter referred to by the cultivar name ‘Danat213’.

The new cultivar ‘Danat213’ is a product of a planned mutant selection and monitoring program in Moshav Mishmar Hashiva, Israel, with the objective of finding color mutants of the Chrysanthemum morifolium cultivar ‘Danchryat’.

The new cultivar ‘Danat213’ originated from an induced mutation on a single flowering plant of the Chrysanthemum morifolium cultivar ‘Danchryat’ (patented, U.S. Plant Pat. No. 15,045), and grown in a controlled, cultivated environment in Moshav Mishmar Hashiva, Israel. The parental cultivar ‘Danchryat’ is also known by the commercial variety name Atlantis.

The new cultivar ‘Danat213’ was discovered and selected by the inventor, Gabriel Danziger, as an induced, whole plant mutation of ‘Danchryat’ in 2003 in Moshav Mishmar Hashiva, Israel. The mutation arose by radiation.

Asexual reproduction of the new cultivar by stem cutting was first performed in January of 2004 in Moshav Mishmar Hashiva, Israel, and has demonstrated that the combination of characteristics as herein disclosed for the new cultivar are firmly fixed and retained through successive generations of asexual reproduction. The new cultivar reproduces true to type.

### BRIEF SUMMARY OF THE INVENTION

The following traits have been repeatedly observed and are determined to be unique characteristics of ‘Danat213’ which in combination distinguish this Chrysanthemum as a new and distinct cultivar:

1. Inflorescence with oblong shaped, yellow ray florets, and bell shaped, yellow disc florets;

2. Flowering time of 7.5 weeks;

3. Short day plant; and

4. Vigorous growth habit.

Plants of the new cultivar ‘Danat213’ differ from plants of the parental cultivar ‘Danchryat’ as described in Table 1.

TABLE 1

| | | ‘Danchryat’ |
|---|---|---|
| Characteristic | ‘Danat213’ | (patented, PP15,045) |
| Inflorescence Diameter | 9.5 cm. | 9.5 cm. |
| Flowering Time | 7.5 weeks | 7.5 weeks |
| Growth Potency | Medium-High | Medium-High |
| Ray Floret Color | Yellow, RHS 4-C | White, RHS 155-D |
| Disk Floret Color (when | Yellow green, | Yellow green, |
| opening) | RHS N144-B | RHS 146-B |

Of the many commercial cultivars known to the present inventor, the most similar in comparison to ‘Danat213’ is the parental cultivar ‘Danchryat’ as described in Table 1.

### BRIEF DESCRIPTION OF THE DRAWINGS

The accompanying photographs illustrate the overall appearance of the new Chrysanthemum cultivar ‘Danat213’ showing the colors as true as is reasonably possible with colored reproductions of this type. Colors in the photographs may differ slightly from the color values cited in the detailed botanical description, which accurately describe the colors of ‘Danat213’.

FIG. 1 shows a side view perspective of a corymb of ‘Danat213’. FIG. 2 shows a close-up view of an inflorescence of ‘Danat213’.

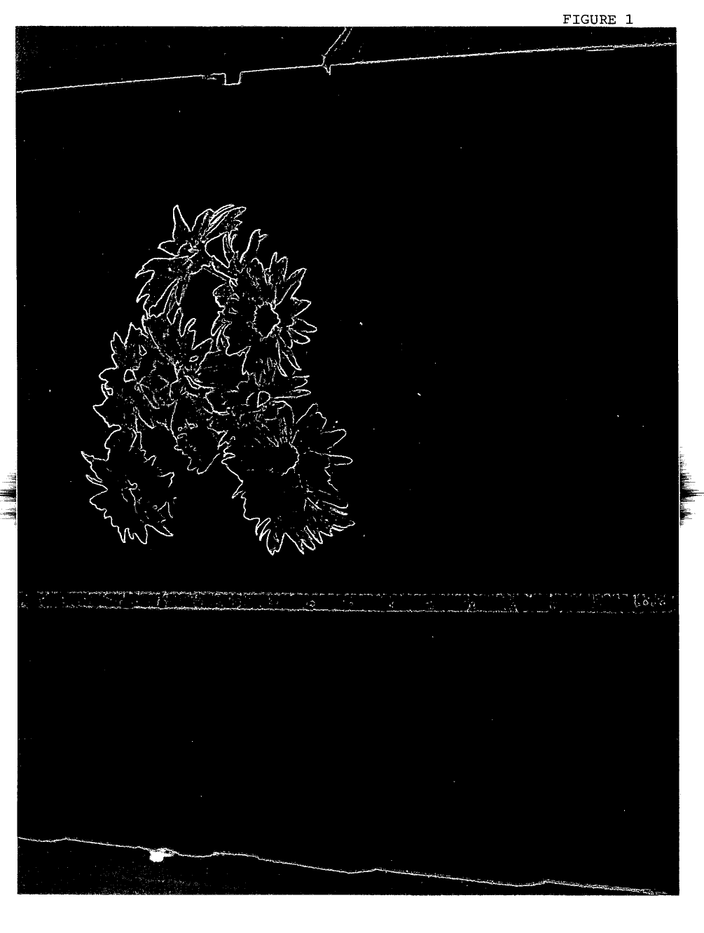

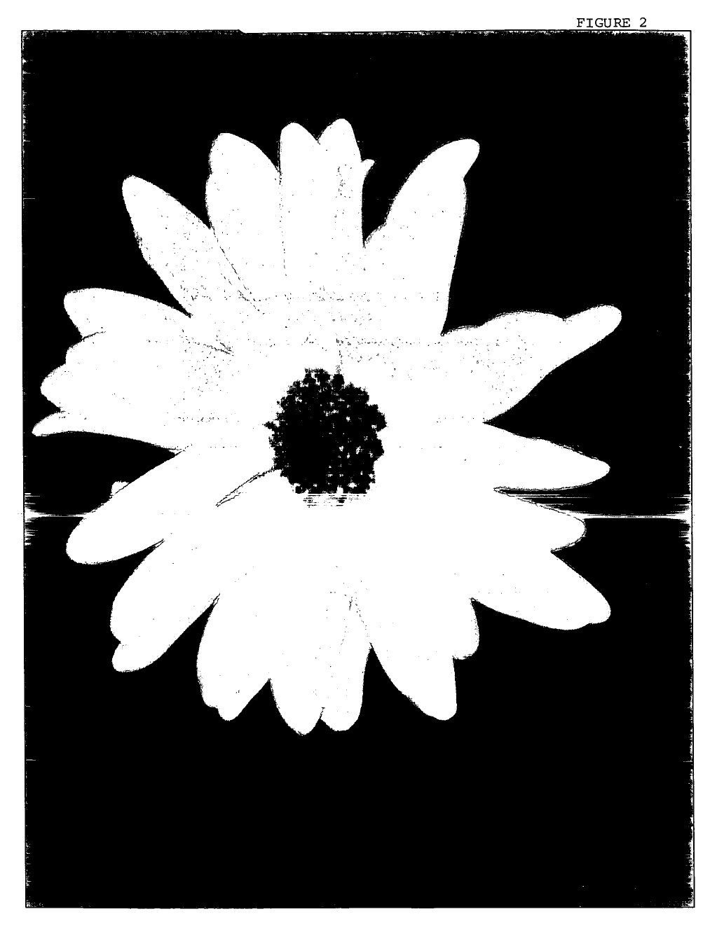

### DETAILED BOTANICAL DESCRIPTION

‘Danat213’ has not been observed under all possible environmental conditions. The phenotype of the new cultivar may vary with variations in environment such as temperature, light intensity, and daylength without any change in the genotype of the plant.

The aforementioned photographs, together with the following observations, measurements and values describe the new cultivar ‘Danat213’ as grown in a greenhouse in Moshav Mishmar Hashiva, Israel, under conditions which closely approximate those generally used in commercial practice. Plants of ‘Danat213’ are mainly grown in greenhouses, but can also be grown outdoors during the summer. The recommended temperature range for growing plants of ‘Danat213’ in a greenhouse are: day temperature range of 22° C. to 23° C., and evening temperature range of 17° C. to 18° C. For premium growth conditions, plants of ‘Danat213’ are irrigated and fertilized on a regular basis using moderate regim. Plants of ‘Danat213’ are treated with the growth retardant, Alar (B-Nine), at a concentration of 3 gr/L.

Color references are made to The Royal Horticultural Society Colour Chart (RHS,) ed. 2001, except where general colors of ordinary significance are used. Color values were taken under daylight conditions at approximately 12:00 noon in Moshav Mishmar Hashiva, Israel. The age of the plant described is 11 weeks from planting.
Classification:
   Botanical.—Chrysanthemum morifolium.
   Commercial.—Chrysanthemum cv. ‘Atlantis Lemon’.
Parentage:
   Parental cultivar.—Induced mutation of Chrysanthemum morifolium cultivar ‘Danchryat’ (patented, U.S. Plant Pat. No. 15,045).
Propagation: Stem cutting.
Plant:
   General Appearance and Form.—Height: 90 cm Width: 20cm Shape: Compound corymbiform.
   Flowering response.—Short day type.
   Flowering season.—Autumn and beginning of winter.
   Winter hardiness.—Hardy in moderate climate.
   Lastingness of the individual bloom.—3 to 3.5 weeks (on the stem).
   Rooting habit.—Adventitious roots from cutting base.
   Time to initiate roots.—6 to 7 days.
   Fragrance.—None.
Foliage:
   Leaf length.—10 cm.
   Leaf width.—5.5 cm.
   Overall Shape of Leaf.—Pinnatipartite.
   Apex Shape.—Rounded.
   Base Shape.—Wedge-shaped.
   Margin.—Serrate.
   Texture.—Slightly warty.
   Color of Upper Surface.—Mature leaf: Yellow-green, RHS 147-A Immature leaf: Yellow-green, RHS 147-A.
   Color of Lower Surface.—Mature leaf: Yellow-green, RHS 147-B Immature leaf: Yellow-green, RHS 147-B.
   Venation.—Pattern: Pinnate Color: Upper surface: Yellow-green 147-B Lower surface: Yellow-green 147-C.
   Petiole.—Length: 2 cm Diameter: 4 mm Color: Yellow-green, RHS 146-A.
   Stem.—Average length: 85 to 90 cm Average diameter: 8 to 10 mm Color: Yellow-green, RHS 146-B Internode length: 15 to 20 mm.
Inflorescence:
   Flowering response.—Short day.
   Quantity of inflorescences on average.—7 to 8 on average per stem.
   Inflorescence bud.—Height: 1 cm Diameter: 16 mm Color: Yellow-green, RHS 146-B.
   Inflorescence size.—Diameter: 9.5 cm Depth (height): 18 to 20 mm Diameter of disc: 18 mm.
   Ray florets.—Number of ray florets per inflorescence: 30 to 32 Orientation: Upward Length: 35 to 40 mm Width: 12 to 13 mm Apex width: 4 to 8 mm Overall shape: Oblong Apex shape: Rounded (obtuse) Base shape: Fused Margin: Entire Texture: Smooth Color when opening: Yellow group, RHS 4-C Color when fully opened: Upper surface: Yellow group, RHS 4-C Lower surface: Yellow group, RHS 5-C.
   Disc florets.—Number of disc florets per inflorescence: 200 to 220. Length: 4 to 5 mm Width: 1 mm Overall shape: Bell shaped (campanulate) Apex shape: Toothed Base shape: Fused Margin: Entire Texture: Smooth Color when opening: Yellow-green, RHS N144-B Color when fully opened: Upper surface: Yellow, RHS 9-A Lower surface: Yellow, RHS 9-C.
   Phyllaries.—Average number: 15 to 18 Average length: 6 to 9 mm Average width: 1 to 2 mm Overall shape: Oblong Apex shape: Rounded (obtuse) Base shape: Truncate Margin: Entire Color: Upper surface: Yellow green, RHS 148-B Lower surface: Yellow green, RHS 144-A.
   Peduncles.—Aspect: Upward Length: 15 cm Diameter: 3.5 to 4 mm Texture: Slightly warty Color: Yellow-green, RHS 146-B.
Reproductive organs:
Androecium: Present in disc florets only.
   Stamen.—Number: 5, fused as a tube. Length: 5 mm Color: Yellow, RHS 13-A.
   Anthers.—Color: Yellow, RHS 13-A.
   Pollen.—Color: Yellow, RHS 13-A.
Gynoecium: Present in both ray and disc florets.
   Style.—Length: 5 to 6 mm Color: Yellow, RHS 2-B.
   Stigma.—Width: 0.3 mm Color: Yellow, RHS 12-B.
   Ovary.—Color: Yellow-green, RHS 149-D.
   Seeds.—Length: 1.5 mm Width: 0.8 to 1 mm Shape: Oblong Color: Grayed-green 197-A.
   Fruit.—Shape: Achene Color: Grayed-green, RHS 197-A.
Disease resistance/susceptibility: None observed under regular growing conditions.

I claim:

1. A new and distinct Chrysanthemum plant named ‘Danat213’, substantially as illustrated and described herein.
